(19) United States
(12) Patent Application Publication
(10) Pub. No.: US 2008/0184510 A1
(43) Pub. Date: Aug. 7, 2008

(54) Products for Tongue Cleaning and for Preventing and Treaing Halitosis and Equipment for Tongue Cleaning

(76) Inventor: Mauricio Duarte Da Conceicao, Sao Paulo (BR)

Correspondence Address: AMIN, TUROCY & CALVIN, LLP, 1900 EAST 9TH STREET, NATIONAL CITY CENTER, 24TH FLOOR,, CLEVELAND, OH, 44114, US

(21) Appl. No.: 11/916,270

(22) PCT Filed: Jun. 2, 2006
(86) PCT No.: PCT/BR06/00108

§ 371 Date: Nov. 30, 2007

(30) Foreign Application Priority Data
Jun. 3, 2005 (BR) PI 0502144-8

(51) Int. Cl. A61K 8/22; A61B 17/24; A46B 15/00; A46B 9/04; A61Q 11/00

(52) U.S. Cl. 15/106; 424/53; 606/161; 15/167.1

(57) ABSTRACT

The described aspects relate to products heretofore unknown by the art, and tongue cleaning equipment for preventing and treating halitosis, comprising a gel/cream that in one aspect comprises Hydrogen peroxide, Citric acid, flavorizer, demineralized water, Cetylpyridinium chloride, colorant, Sodium saccharine, Sorbitol and Poloxamer, or a spray that in one aspect comprises Hydrogen peroxide, Citric acid, flavorizer, demineralized water, Cetylpyridinium chloride, colorant, Sodium saccharine, Sorbitol, Polisorbate 20 (Tween 20), Sodium benzoate, and Methyl paraben, in adequate proportions, and the equipment comprises a device to detach tongue coating, comprised of a toothbrush or scraper provided with a brush employed to brush the tongue, along with a portion of the gel/cream or spray, applied over the bristles of the brush or scraper, and a device to remove the detached tongue coating.

### FIELD OF THE INVENTION

The present invention relates to the field of oral hygiene products, particularly the ones helping treatment and control of bad breath. Through its effect, it is possible to facilitate the removal and diminish the new formation of tongue coating and reduce the concentration and formation of volatile sulfur compounds.

### BACKGROUND OF THE INVENTION

Bad breath (whose medical term is halitosis) affects more than 30% of the Brazilian population and research data in other countries shows similar indexes worldwide.

The most frequent cause of bad breath is found in the oral cavity and is called tongue coating, present in more than 90% of the cases.

Tongue coating is a bacterial plaque, whitish, or yellowish, which forms on the posterior part of the tongue. It is composed of epithelial cells peeled off the oral mucosa, salivary proteins and food protein remains, which will serve as food substratum to existing bacteria (which are anaerobic proteolytic bacteria). As the bacterial metabolism ends, bad smelling compounds are produced, called Volatile Sulfur Compounds (VSCs), which are gases derived from sulfur: Sulfur hydrides (SH₂), Methyl mercaptanes (CH₃SH), and Dimethyl sulfides [(CH₃)₂ S].

Such compounds, once they reach a certain concentration, provide breath a characteristic smell of sulfur or rotten egg, which is nothing but bad breath itself.

Tongue coating is essentially formed when one faces a reduction of salivary flow or an epithelial desquamation above physiological limits, or even both situations. There are several causes for increasing cell desquamation, among them desiccation caused by oral respiration or snoring, frequent ingestion of alcoholic beverages or even the use of mouthwashes containing alcohol, the use of orthodontic appliances and the habit of biting lips and cheeks, among others. A reduction in saliva occurs mainly due to stress and use of medications that diminish the saliva production as a side effect, as well as the low ingestion of liquids, besides other less frequent causes.

Products used in preventing and treating bad breath, such as, for example, those used for mouth rinsing or gargling, or sprays to improve mouth breath, normally use alcohol in their formulation. This fact leads to the dehydration and desiccation of the oral mucosa, increasing cell desquamation, and hence, increasing the formation of tongue coating, since proteolytic bacteria shall decompose these desquamated cells, originating volatile sulfur compounds (VSCs) in such process. Furthermore, many types of toothpaste contain the detergent sodium lauryl sulfate or similar ingredients, which may also desquamate the oral mucosa. Thus, a product intended for treating and/or controlling bad breath cannot contain alcohol or sodium lauryl sulfate or similar ingredients in its formulation.

Another important factor is that, among bacteria present in tongue coating, there are bacteria that may cause systemic diseases, such as, for example, gastritis and pneumonia, as well as may cause tooth cavities and periodontal disease, the latter of which also causes a number of systemic diseases. Therefore, removal of tongue coating and the control of its formation, are important tools for preventing tooth cavities, periodontal disease, and systemic diseases.

### SUMMARY OF THE INVENTION

The invention may include a compound, in the form of gel/cream or spray, developed to facilitate the “detaching” and subsequent removal of tongue coating, the decreasing of its new formation and the reduction of concentration and formation of volatile sulfur compounds, heretofore unknown to the art.

In this context, gel/cream and spray are comprised of ingredients common to both, basically comprising: a) active ingredient; b) pH adjusters; c) flavorizer; d) bactericide; e) colorant; f) edulcorant; g) solubilizer media, and specific ingredients for gel/cream: h) thickener; and i) physical abrasive and, for the spray: h) preservative; and i) solubilizer.

Regarding tongue coating, the active ingredient group is selected to have, as its main effect, the oxygenating of the microscopic spaces existing between lingual papillae, by using a tongue scraper that has a brush, or by using a toothbrush, facilitating the “detaching” of tongue coating and its subsequent removal, which will be accomplished by using a tongue scraper. Furthermore, through its residual effect, the active ingredient also contributes to decrease new tongue coating formation.

The pH adjustment group is selected to allow determining the product's pH, and also works as a hydrogen peroxide stabilizer, which is part of the compound.

The flavorizer group is selected so as to have, as its main effect, giving the product a pleasant flavor and hence, providing the mouthwash user's breath with a pleasant scent.

The bactericide group is selected to allow a reduction in the accumulation of bacterial plaque and to prevent gingivitis.

The colorant group is selected to allow coloring the product.

The edulcorant group is selected to allow sweetening the product, giving it a pleasant taste to the palate.

The solubilizer media group is selected to work as a base for all ingredients present in the formulation.

The thickener group comprises substances selected to be added to products to give them a jelly or creamy consistency.

The preservative group is selected among substances that are added to personal hygiene products, cosmetics and perfumes, whose primary purpose is to preserve them from damage and/or deterioration caused by microorganisms during manufacture and storage, as well as to protect the consumer from inadvertent contamination during use of the product.

The group of physical abrasives is comprised of selected substances added to products intended to increase abrasion and consequently allow a larger cleaning effect. Its use in the formulation of gel/cream is optional, whenever a more abrasive product is wanted.

The solubilizer group is selected to greatly facilitate the miscibilization of flavorizer with solubilizer media.

Thus, the preferred composition for gel/cream essentially contains: Hydrogen peroxide, Citric acid, flavorizer, demineralized water, Cetylpyridinium chloride, colorant, Sodium saccharine, Sorbitol and Poloxamer.

The preferred composition of spray essentially contains: Hydrogen peroxide, Citric acid, flavorizer, demineralized water, Cetylpyridinium chloride, colorant, Sodium saccharine, Sorbitol, Polisorbate 20 (Tween 20), Sodium benzoate and Methyl paraben.

The way to use the products is by applying them (as gel/cream or spray) directly onto the toothbrush or tongue scraper that has a brush.

### DETAILED DESCRIPTION OF THE INVENTION

The formulations of the present invention contain at least nine different types of ingredients, including, but not limited to, ingredient types with an intense oxidizing effect due to the oxygen that is freed, ingredient types with a flavorizing effect and ingredient types with a bactericide effect.

In order to obtain ideal appearance and flavors, there are also edulcorant, solubilizer, and colorant ingredients. Some edulcorants may also have a thickening effect.

In order to facilitate the product's use and stability, pH adjusters and preservatives are provided.

These ingredients are contained in aqueous-base media so that active agents may be conducted to the oral cavity.

In order to provide the product a jelly or creamy consistency, a type of ingredient with a thickening effect is provided.

In order to increase the cleansing power, a type of ingredient with an abrasive effect is provided.

The group of ingredients having a strong oxidant effect due to oxygen liberation includes, but is not limited to, Hydrogen peroxide, Monohydrate sodium perborate and Chlorine dioxide (Sodium chlorite/Sodium chlorate). Preferably, the ingredient having a strong oxidant effect is hydrogen peroxide.

The group of ingredients having a strong oxidant effect is present in the formulation in a quantity of about 0.05% of the total volume to about 3% of the volume, preferably about 0.1% of the volume to about 2% of the volume, preferably about 0.5% of the volume to 1% of the volume, and preferably about 0.7% of the volume of whole formulation.

The group of ingredients facilitating the use and the stability of the product includes, but it is not limited to, pH adjusters and preservatives.

The pH adjuster group includes, but is not limited to, Boric acid and Citric acid. Preferably, the pH adjuster is Citric acid.

The group of ingredients having a pH adjusting effect is present in the formulation in a quantity of about 0.01% of the total volume to about 1% of the volume, preferably about 0.05% of the volume to about 0.5% of the volume, preferably about 0.1% of the volume to about 0.25% of the volume, and preferably about 0.15% of the volume of the whole formulation.

The ingredient having a preservative effect includes, but is not limited to, Sodium benzoate, Nipagin or Methyl paraben, Benzoic acid, Formaldehyde, Thymol and Nipazol or Isopropyl paraben. Preferably, the preservatives are Sodium benzoate and Methyl paraben.

The group of ingredients having a preservative effect, more specifically Methyl paraben, is present in the formulation in a quantity of about 0.01% of the total volume to about 0.5% of the volume, preferably about 0.02% of the volume to about 0.25% of the volume, preferably about 0.03% of the volume to about 0.1% of the volume, and preferably about 0.05% of the volume of the whole formulation.

The group of ingredients having a preservative effect, more specifically Sodium benzoate, is present in the formulation in a quantity of about 0.01% of the total volume to about 1% of the volume, preferably about 0.05% of the volume to about 0.5% of the volume, preferably about 0.1% of the volume to about 0.25% of the volume, and preferably about 0.15% of the volume of the whole formulation.

The ingredient group having a bactericide effect includes, but is not limited to, Cetylpyridinium chloride, Delmopinol, Benzalkonium chloride, Sodium bicarbonate, Chlorhexidine gluconate; Chlorhexidine digluconate, Chlorine dioxide (Sodium chlorite/Sodium chlorate), Triclosan, Biguanide chlorhydrate, Sanguinaria canadensis, Propolis, Aloe vera, Sage (Salvia officinalis), Lemon (Citrus limon), Pine (Pinus sylvestris), Echinacea (Echinacea purpurea and angustifolia), Rathany (Krameria trianda) and Cheeseweed mallow (Malva parviflora L.). Preferably, the bactericide ingredient is cetylpyridinium chloride.

The ingredient group having a bactericide effect is present in the formulation in a quantity of about 0.01% of the total volume to about 0.5% of the volume, preferably about 0.02% of the volume to about 0.25% of the volume, preferably about 0.03% of the volume to about 0.1% of the volume, and preferably about 0.05% of the volume of the whole formulation.

The group of ingredients having a flavorizing effect includes, but is not limited to, all aromatizers for oral hygiene products available in the market, including Zinc citrate, Zinc chloride, Methyl salicylate, eucalyptus oil, spearmint oil, and peppermint oil.

Preferably, a variety of ingredients with a flavorizer effect are used to allow for a broad range of flavor options.

The ingredient group having a flavorizing effect is present in the formulation in a quantity of about 0.01% of the total volume to about 1% of the volume, preferably about 0.05% of the volume to about 0.5% of the volume, preferably about 0.075% of the volume to about 0.3% of the volume, and preferably about 0.125% of the volume of the whole formulation.

The group of ingredients used to obtain ideal appearance and flavor includes, but it is not limited to, ingredients with an edulcorant, solubilizer, and colorant effect.

The ingredient having an edulcorant effect includes, but is not limited to, Sodium saccharine, Sorbitol, Xylitol, Aspartame, Sodium cyclamate, and Stevia. Preferably, the ingredients having an edulcorant effect are Sodium saccharine and Sorbitol.

The group of ingredients having an edulcorant effect, more specifically Sodium saccharine, is present in the formulation in a quantity of about 0.01% of the total volume to about 1% of the volume, preferably about 0.04% of the volume to about 0.5% of the volume, preferably about 0.08% of the volume to about 0.25% of the volume, and preferably about 0.125% of the volume of the whole formulation.

The group of ingredients having an edulcorant effect, more specifically Sorbitol, only to be used in this percentage amount in a spray for tongue cleansing, is present in the formulation in a quantity of about 0.5% of the total volume to about 20% of the volume, preferably about 1% of the volume to about 15% of the volume, preferably about 3% of the volume to about 10% of the volume, and preferably about 5% of the volume of the whole formulation.

The group of ingredients having an edulcorant effect, more specifically Sorbitol, only to be used in this percentage amount in gel/cream for tongue cleansing, is present in the formulation in a quantity of about 2.5% of the total volume to about 75% of the volume, preferably about 7.5% of the volume to about 50% of the volume, preferably about 12.5% of the volume to about 25% of the volume, and preferably about 15% of the volume of the whole formulation.

The group of ingredients having a solubilizer effect includes, but it is not limited to, Polisorbate 20 (Tween 20), Propylene glycol, Polyoxyl 40 and a solubilizer that mixes Propylene glycol, Polyethylene glycol and water. Preferably, the solubilizer ingredient is Polisorbate 20.

The group of ingredients having a solubilizer effect is present in the formulation in a quantity of about 0.05% of the total volume to about 2% of the volume, preferably about 0.1% of the volume to about 1.5% of the volume, preferably about 0.25% of the volume to about 1% of the volume, and preferably about 0.5% of the volume of the whole formulation.

The group of ingredients having a colorant effect includes, but is not limited to, all colorants for food or oral hygiene products available in the market. Preferably, different ingredients with a colorant effect are used to provide the product with a range of color options.

The group of ingredients having a colorant effect is present in the formulation in a quantity of about 0.0001% of the total volume to about 1% of the volume, preferably about 0.0005% of the volume to about 0.1% of the volume, preferably about 0.001% of the volume to about 0.01% of the volume, and preferably about 0.002% of the volume of the whole.

The group of ingredients enabling active agents to be conducted to the oral cavity in an aqueous-base solubilizer media includes, but is not limited to, demineralized water, distilled water, deionized water, and mineral water.

Preferably, the aqueous-base solubilizer media comprises demineralized water.

The group of thickeners used to give the product a jelly or creamy consistency includes, but is not limited to, Carbopol, Sodium carboxymethylcellulose, Xantan gum, Hydrated silica and Poloxamer. Preferably, the thickening ingredient comprises Poloxamer.

The ingredient group having a thickening effect is present in the formulation in a quantity of about 0.1% of the total volume to about 60% of the volume, preferably about 3% of the volume to about 45% of the volume, preferably about 9% of the volume to about 30% of the volume, and preferably about 18% of the volume of the whole formulation.

The ingredient group that gives the product an abrasive effect includes, but is not limited to, Silica, Silicon dioxide, Dicalcic phosphate, Calcium pyrophosphate, Zinconium silicate, Sodium bicarbonate and Calcium carbonate. Preferably, the physical abrasive ingredient comprises Silica.

The group of ingredients with an abrasive effect is present in the formulation in a quantity of about 0.1% of the total volume to about 12% of the volume, preferably about 0.5% of the volume to about 8% of the volume, preferably about 1% of the volume to about 4% of the volume, and preferably about 2% of the volume of the whole formulation.

The invention shall be more clearly understood with the following examples, merely intended to exemplify, not to limit the invention.

### EXAMPLES OF GEL/CREAM FORMULATION

### Example 1

| Ingredient | Dosage | Function |
|---|---|---|
| Citric acid | 0.15% | Stabilizer |
| Demineralized water | q.s.p. | Solubilizer Media |
| Flavorizer | 0.125% | Flavor |
| Sodium carboxymethylcellulose | 0.30% | Thickener |
| Cetylpyridinium chloride | 0.05% | Bactericide |
| Colorant | 0.002% | Colorant |
| Hydrogen peroxide | 0.7% | Active |
| Sodium saccharine | 0.125% | Edulcorant |
| Sorbitol | 70.00% | Edulcorant |
| Hydrated Silica | 14.00% | Thickener |
| Syloid 63 (silica) | 2.00% | Physical Abrasive |

### Example 2

| Ingredient | Dosage | Function |
|---|---|---|
| Citric acid | 0.15% | Stabilizer |
| Demineralized water | q.s.p. | Solubilizer Media |
| Flavorizer | 0.125% | Flavor |
| Cetylpyridinium chloride | 0.05% | Bactericide |
| Colorant | 0.002% | Colorant |
| Hydrogen peroxide | 0.7% | Active |
| Poloxamer | 18.0% | Thickener |
| Sodium saccharine | 0.125% | Edulcorant |
| Sorbitol | 15.00% | Edulcorant |

### Example 3

| Ingredient | Dosage | Function |
|---|---|---|
| Demineralized water | q.s.p. | Solubilizer Media |
| Flavorizer | 0.125% | Flavor |
| Colorant | 0.002% | Colorant |
| Hydrogen peroxide | 0.7% | Active |
| Carbopol | 1.50% | Thickener |
| Sodium saccharine | 0.125% | Edulcorant |
| Sorbitol | 40.00% | Edulcorant |
| Sodium carboxymethylcellulose | 0.30% | Thickener |
| Cetylpyridinium chloride | 0.05% | Bactericide |
| Citric Acid | 0.15% | Stabilizer |

### Example of Spray Formulation

| Ingredients | Dosage | Function |
|---|---|---|
| Citric Acid | 0.15% | pH adjustment |
| Hydrogen peroxide | 0.7% | Active |
| Flavorizer | 0.125% | Flavorizer |
| Sodium benzoate | 0.15% | Preservative |
| Cetylpyridinium chloride | 0.05% | Bactericide |
| Colorant | 0.002% | Colorant |
| Methyl paraben | 0.05% | Preservative |
| Polysorbate 20 (Tween 20) | 0.5% | Solubilizer |
| Sodium Saccharine | 0.125% | Edulcorant |
| Sorbitol | 5% | Edulcorant |
| Demineralized water | q.s.p | Solubilizer Media |

Thus, the preferred composition of gel/cream is: Hydrogen peroxide, Citric acid, flavorizer, demineralized water, Cetylpyridinium chloride, colorant, Sodium saccharine, Sorbitol and Poloxamer, in any quantitative formulation as described above.

The preferred composition of spray is: Hydrogen peroxide, Citric acid, flavorizer, demineralized water, Cetylpyridinium chloride, colorant, Sodium saccharine, Sorbitol, Polisorbate 20 (Tween 20), Sodium benzoate, and Methyl paraben, in any quantitative formulation as described above.

Optionally, instead of the product for tongue cleaning and the prevention and treatment of halitosis with gel/cream or spray, comprising one ingredient of each group, as in the above examples, it may comprise more than one ingredient from each group. Therefore, one of the possibilities would be that the gel/cream or spray composition might have the group of active ingredients formed, for instance, by association with Hydrogen peroxide and Sodium perborate, and/or the group of antimicrobials formed by an association of Cetylpyridinium chloride and Chlorhexidine and/or others.

In other words, the product may comprise one ingredient from each group, as described above, or more than one ingredient from one of the groups, or more than one ingredient from more than one of the groups, or more than one ingredient from all groups, used in association and in appropriate quantities for the desired preventive or treatment effect.

The way to use the product is by applying it (gel/cream or spray) directly onto the toothbrush or tongue scraper that has a brush. Next, one should clean the tongue through the technique described below, especially developed for its use:

1) Make circular movements with the brush on the back part of the tongue, to “detach” tongue coating and to free the product's active principles with the subsequent enhancement of the cleansing effect; and

2) Remove the already “detached” content by scraping the tongue with the tongue scraper, with soft movements from the back to the tip of the tongue.

Important: At the back of the tongue there are structures similar to small “warts” (called circumvallated papillae), visible for the most part. One must be careful not to scrape the tongue scraper over them, thereby injuring them.

Thus, the equipment to perform the treatment according to this invention comprises: 1) a device to “detach” tongue coating, comprising a normal toothbrush (with a set of tufts with bristles; a head containing the implanted tufts of bristles, and a hand piece extending from said head), employed to rub the tongue, and by a portion of the product of this patent, in the form of gel/cream or spray, applied over the bristles to clean the tongue and prevent and treat halitosis; and 2) a device for removing detached tongue coating, comprising a tongue scraper (with at least one edge for scraping the tongue; a structure comprising the edge; and a hand piece attached to the structure).

Optionally, instead of comprising a device to detach tongue coating (brush+product), and a device for removing the detached tongue coating (tongue scraper), said equipment may comprise a single device to accomplish both functions. It is then made up of a tongue scraper that has a brush attached (comprising an edge to scrape the tongue; bristles in opposition to the edge for brushing tongue; a structure containing said edge and brush; and a hand piece attached to said structure), along with a portion of said product applied over the bristles of the scraper.

1.-46. (canceled)

47. A product for tongue cleaning and the prevention and treatment of halitosis comprising a gel/cream or spray comprising ingredients common to both alternatives, made up of at least one ingredient from each of the following ingredient groups: (a) active ingredient, (b) pH adjuster, (c) flavorizer, (d) bactericide, (e) colorant, (f) edulcorant and (g) solubilizer media; and, specific ingredients for gel/cream comprising (h1) thickener and (i1) physical abrasive, or specific ingredients for spray comprising (h2) preservative and (i2) solubilizer;
wherein the:
(a) active ingredient comprises at least one of hydrogen peroxide, or Monohydrate sodium perborate, or Chlorine dioxide (Sodium chlorite/Sodium chlorate);
(b) pH adjuster ingredient comprises at least one of Boric acid or Citric acid;
(c) flavorizer comprises a flavorizer for oral hygiene products including at least one of Zinc citrate, or Zinc chloride, or Tutti frutti, or Menthol, or Methyl salicylate, or eucalyptus oil, or spearmint oil, or peppermint oil;
(d) Bactericide comprises at least one of Cetylpyridinium chloride, or Delmopinol, or Benzalkonium chloride, or Sodium Bicarbonate, or Chlorhexidine gluconate, or Chlorhexidine digluconate, or Chlorine dioxide (Sodium chlorite/Sodium chlorate), or Triclosan, or Polyhexamethylene biguanide chlorhydrate, or Sanguinaria Canadensis, or Propolis, or Aloe Vera, or Sage (Salvia officinalis), or Lemon (Citrus limon), or Pine (Pinus sylvestris), or Echinacea (Echinacea purpurea or angustifolia), or Rathany (Krameria trianda), or Cheeseweed Mallow (Malva parviflora L.);
(e) Colorant comprises at least one of a food colorant product or an oral hygiene colorant product;
(f) Edulcorant comprises at least one of Sodium Saccharine, or Sorbitol, or Xylitol, or Aspartame, or Sodium Cyclamate, or Stevia;
(g) Solubilizer media comprises at least one of demineralized water, or distilled water, or deionized water, or mineral water;
(h1) Thickener in the gel/cream formulation comprises at least one of Carbopol, or Sodium carboxymethylcellulose (SCMC), or Xanthan gum, or Hydrated silica, or Poloxamer;
(h2) Preservative in the spray formulation comprises at least one of Sodium benzoate, or Nipagin, or Methyl paraben, or Formaldehyde, or Benzoic acid, or Formaldehyde, or Thymol, or Nipazol, or Isopropyl paraben;
(i1) Physical abrasive in gel/cream formulation comprises at least one of Silica, or Silicon dioxide, or Dicalcic phosphate, or Calcium pyrophosphate, or Zirconium silicate, or Sodium Bicarbonate, or Calcium Carbonate; and
(i2) Solubilizer in the spray formulation comprises at least one of Polisorbate 20 (Tween 20), or Propylene glycol, or Polyoxyl 40 and a solubilizer mixing Propylene glycol and water or Polyethylene glycol and water.

48. The product of claim 47, wherein the ingredient having a preservative effect (h2) comprises a gel/cream formulation.

49. The product of claim 47, wherein the group of active ingredients (a) having a strong oxidant effect is present in a quantity of about 0.05% of the volume to about 3% of the volume.

50. The product of claim 47, wherein the group of active ingredients (a) having a strong oxidant effect is present in about 0.1% of the volume to about 2% of the volume or from about 0.5% of the volume to about 1% of the volume.

51. The product of claim 47, wherein the group of active ingredients (a) having a strong oxidant effect is present in about 0.7% of the volume of the whole formulation.

52. The product of claim 47, wherein the group of ingredients with a pH adjusting effect (b) is present in a quantity of about 0.01% of the volume to about 1% of the volume.

53. The product of claim 47, wherein the group of ingredients with a pH adjusting effect (b) is present in about 0.05% of the volume to about 0.5% of the volume or in about 0.1% of the volume to about 0.25% of the volume.

54. The product of claim 47, wherein the group of ingredients with a pH adjusting effect (b) is present in about 0.15% of the volume of the whole formulation.

55. The product of claim 47, wherein the group of ingredients having a flavorizing effect (c) is present in a quantity of about 0.01% of the volume to about 1% of the volume.

56. The product of claim 47, wherein the group of ingredients having a flavorizing effect (c), is present in a quantity of about 0.05% of the volume to about 0.5% of the volume or from about 0.075% of the volume to about 0.3% of the volume.

57. The product of claim 47, wherein the group of ingredients having a flavorizing effect (c) is present in a quantity of about 0.125% of the volume of the whole formulation.

58. The product of claim 47, wherein the group of ingredients having a bactericide effect (d) is present in a quantity of about 0.01% of the volume to about 0.5% of the volume.

59. The product of claim 47, wherein the group of ingredients having a bactericide effect (d) is present in a quantity of about 0.02% of the volume to about 0.25% of the volume or from about 0.03% of the volume to about 0.1% of the volume.

60. The product of claim 47, wherein the group of ingredients, having a bactericide effect (d), is present in a quantity of about 0.05% of the volume of the whole formulation.

61. The product of claim 47, wherein the group of ingredients having a colorant effect (e) is present in a quantity of about 0.0001% of the volume to about 1% of the volume.

62. The product of claim 47, wherein the group of ingredients having a colorant effect (e) is present in a quantity of about 0.0005% of the volume to about 0.1% of the volume or from about 0.001% of the volume to about 0.01% of the volume.

63. The product of claim 47, wherein the group of ingredients having a colorant effect (e), is present in a quantity of about 0.002% of the volume of the whole formulation.

64. The product of claim 47, wherein the group of ingredients having an edulcorant effect (f) comprises Sodium Saccharine present in a quantity of about 0.01% of the volume to about 1% of the volume.

65. The product of claim 64, wherein the Sodium Saccharine is present in a quantity of about 0.04% of the volume to about 0.5% of the volume, or from about 0.08% of the volume to about 0.25% of the volume.

66. The product of claim 64, wherein the Sodium Saccharine is present in a quantity of about 0.125% of the volume of the whole formulation.

67. The product of claim 47, wherein the group of ingredients having an edulcorant effect (f), comprises Sorbitol, which when used in the spray for tongue cleaning, is present in a quantity of about 0.5% of the volume to about 20% of the volume.

68. The product of claim 67, wherein the Sorbitol is present in an amount from about 1% of the volume to about 15% of the volume or from about 3% of the volume to about 10% of the volume.

69. The product of claim 67, wherein the Sorbitol is present in a quantity of about 5% of the volume of the whole formulation.

70. The product of claim 47, wherein the group of ingredients having an edulcorant effect (f) comprises Sorbitol, which when used for gel/cream for tongue cleaning, is present in a quantity of about 2.5% of the total volume to about 75% of the volume.

71. The product of claim 70, wherein the Sorbitol is present in about 7.5% of the volume to about 50% of the volume, or from about 12.5% of the volume to about 25% of the volume.

72. The product of claim 70, wherein the Sorbitol is present in a quantity of about 15% of the volume of the whole formulation.

73. The product of claim 47, wherein the solubilizer media (g) comprises an aqueous-base solubilizer media including demineralized water.

74. The product of claim 47, wherein the Thickener (h1) is present in a quantity of about 0.1% of the total volume to about 60% of the volume.

75. The product of claim 47, wherein the Thickener (h1) is present in a quantity of about 3% of the volume to about 45% of the volume, or about 9% of the volume to about 30% of the volume.

76. The product of claim 47, wherein the Thickener (h1) is present in a quantity of about 18% of the volume of the whole formulation.

77. The product of claim 47, wherein the Physical Abrasive (i1) is present in a quantity of about 0.1% of the total volume to about 12% of the volume.

78. The product of claim 47, wherein the Physical Abrasive (i1) is present in a quantity of about 0.5% of the volume to about 8% of the volume, or about 1% of the volume to about 4% of the volume.

79. The product of claim 47, wherein the Physical Abrasive (i1) is present in a quantity of about 2% of the volume of the whole formulation.

80. The product of claim 47, wherein the Preservative (h2) comprises Methyl paraben present in a quantity of about 0.01% of the volume to about 0.5% of the volume.

81. The product of claim 80, wherein the Methyl paraben is present in a quantity of about 0.02% to about 0.25% of the volume, or about 0.03% to about 0.1% of the volume.

82. The product of claim 80, wherein the Methyl paraben is present in a quantity of about 0.05% of the volume of the whole formulation.

83. The product of claim 47, wherein the preservative (h2) comprises Sodium benzoate present in a quantity of about 0.01% of the volume to about 1.0% of the volume.

84. The product of claim 83, wherein the Sodium benzoate is present in a quantity of about 0.05% of the volume to about 0.5% of the volume, or about 0.1% of the volume to about 0.25% of the volume.

85. The product of claim 83, wherein the Sodium benzoate is present in a quantity of about 0.15% of the volume of the whole formulation.

86. The product of claim 47, wherein the solubilizer (i2) is present in a quantity of about 0.05% of the total volume to about 2% of the volume.

87. The product of claim 47, wherein the solubilizer (i2) is present in a quantity of about 0.1% of the volume to about 1.5% of the volume, or about 0.25% of the volume to about 1% of the volume.

88. The product of claim 47, wherein the solubilizer (i2) is present in a quantity of about 0.5% of the volume of the whole formulation.

89. The product of claim 47, wherein the gel/cream comprises Hydrogen peroxide, Citric acid, flavorizer, demineralized water, Cetylpyridinium chloride, colorant, Sodium saccharine, Sorbitol and Poloxamer.

90. The product of claim 47, wherein the spray comprises Hydrogen peroxide, Citric acid, flavorizer, demineralized water, Cetylpyridinium chloride, colorant, Sodium saccharine, Sorbitol, Polisorbate 20 (Tween 20), Sodium benzoate and Methyl paraben.

91. Equipment for tongue cleaning, comprising:
a device to “detach” tongue coating, comprising a toothbrush employed to rub the tongue, and a portion of the product to clean the tongue and to prevent and treat halitosis according to claim 47, in the form of gel/cream or spray, operable to be applied over the bristles; and
a device for removing the “detached” tongue coating comprising a tongue scraper.

92. The equipment of claim 45, wherein the device to “detach” tongue coating and the device for removing the “detached” tongue coating comprise a single device.
